(19) United States
(12) Patent Application Publication
(10) Pub. No.: US 2025/0214067 A1
(43) Pub. Date: Jul. 3, 2025

(54) CATALYSTS AND PROCESS FOR CATALYTICALLY CONVERTING NAPHTHA TO LIGHT OLEFINS

(71) Applicant: UOP LLC, Des Plaines, IL (US)

(72) Inventors: Deng-Yang Jan, Elk Grove Village, IL (US); Mathias Paul Koljack, Glenview, IL (US); Gregory A. Funk, Carol Stream, IL (US); Raelynn M Miller, La Grange, IL (US); Jaime G. Moscoso, Mount Prospect, IL (US)

(21) Appl. No.: 18/977,767

(22) Filed: Dec. 11, 2024

Related U.S. Application Data
(60) Provisional application No. 63/616,650, filed on Dec. 31, 2023.

(51) Int. Cl. B01J 29/44; B01J 37/00; B01J 37/02; B01J 37/12; B01J 37/30; C07C 5/03; C07C 5/32

(52) U.S. Cl. CPC B01J 29/44; B01J 37/0009; B01J 37/0207; B01J 37/0236; B01J 37/12; B01J 37/30; C07C 5/03; C07C 5/325; B01J 2229/18; B01J 2229/36; C07C 2523/42

(57) ABSTRACT

A zeolitic catalyst and a process for its use is provided where the metal, preferably platinum, on the catalyst is not well dispersed as shown by low levels of H2 chemisorption, and low chemisorption hydrogen to platinum ratios. In a process for converting naphtha to light olefins that comprises contacting a naphtha stream with a zeolitic catalyst to produce a light paraffin stream at conditions which dehydrogenate the naphtha to olefins, interconvert the olefins to lighter olefins and hydrogenate the lighter olefins to produce a light paraffin stream comprising ethane and propane, this catalyst produces a higher proportion of ethane.

### FIELD

The field is the conversion of naphtha to light olefins. The field may particularly relate to converting naphtha to light olefins in a two-step conversion process. The field may more specifically relate to the use of a catalyst that maximizes production of ethylene.

### BACKGROUND

Light olefin production is vital to the production of sufficient plastics to meet worldwide demand. Paraffin dehydrogenation (PDH) is a process in which light paraffins such as propane and butane can be dehydrogenated to make propylene and butylene, respectively. Dehydrogenation is an endothermic reaction which requires external heat to drive the reaction to completion.

Fluid catalytic cracking (FCC) is another endothermic process that can be tuned to produce substantial propylene. However, not every FCC unit is tuned to make substantial propylene. Also, high propylene FCC units do not make much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to the cracking furnace to reduce the hydrocarbon partial pressure and enhance olefin yield and to reduce the formation and deposition of carbonaceous material in the cracking reactors. The process is therefore often referred to as steam cracking or pyrolysis. Ethane oxidative dehydrogenation is a newer catalytic process for converting ethane to ethylene which can be conducted at lower temperatures with lower carbon oxide emissions than steam cracking.

Two types of feeds are typically used for steam cracking. Ethane feed is used in regions where light hydrocarbon gases are prevalent. In regions, where gas is not abundant, naphtha feed is employed for steam cracking. Pyrolytic naphtha cracking has long set the price in the ethylene industry due to higher production cost versus pyrolytic ethane cracking. The world does not currently produce enough ethane to supply the growing demand for ethylene. Therefore, regions lacking ethane supply such as Asia and Europe rely mainly on naphtha cracking to supply ethylene. Naphtha cracking yields only about 30%-35% ethylene with the balance including both high-value by-products comprising propylene, butadiene, and butene-1 and relatively low value by-products comprising pyoil, pygas, and fuel gas. Additional pressures on naphtha cracking including minimum production requirements and environmental concerns have led to the withholding of government approvals in certain regions such as China. The ethylene industry needs a more efficient, economical and environmentally friendly route to light olefins from naphtha feeds.

Naphtha to ethane and propane (NEP), when coupled with ethane steam cracker and propane dehydrogenation, is an efficient on-purpose ethylene and propylene production pathway, with great improvement in molecular transformation efficiency. To enable dominant ethane production (high ethane to propane), matching a greater demand for ethylene (over propylene), to maximize ethylene production, it is required of NEP to produce high ethane and low propane. This disclosure provides a catalyst having attributes to produce both a high level of ethane to propane and low amounts of methane.

### BRIEF SUMMARY

A process for converting naphtha to light olefins comprises contacting a naphtha stream with a catalyst to produce a light paraffin stream at conditions which dehydrogenate the paraffins to olefins, interconvert the olefins to lighter olefins and hydrogenate the lighter olefins to produce a light paraffin stream comprising ethane and propane. It has been found that a catalyst having the following properties can be used to produce a greater amount of ethylene than other catalysts. In general, it is assumed that a metal deposited upon a catalyst provides a higher yield or greater selectivity when the metal is well dispersed. However, in this disclosure it has been found that a less dispersed metal produces a higher yield of ethane as desired herein. The dispersion is indicated by the amount of hydrogen that is adsorbed by the catalyst with lower amounts of hydrogen adsorbed when the metal is not well dispersed. The catalyst contains more than 0.02 wt % platinum and has more than about 0.15 mmol/gram acidity. This catalyst has a low ratio of chemisorption of hydrogen to platinum and preferably has a metal characteristic of less than 0.45 chemisorption to platinum ratios. The catalyst is further described as having metal attributes of less than 4.5 umoles/gm chemisorption hydrogen.

### BRIEF DESCRIPTION OF THE DRAWINGS

FIG. 1 is a schematic drawing of a process and apparatus of the present disclosure.

FIG. 2 is a schematic drawing of a process and apparatus of an alternative embodiment of FIG. 1.

FIG. 3 is a schematic drawing of a process and apparatus of an additional alternative embodiment of FIG. 1.

FIG. 4 is a schematic drawing of a process and apparatus of a further alternative embodiment of FIG. 1.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx-” refers to molecules that contain less than or equal to x and preferably x and less carbon atoms. The term “Cx+” refers to molecules with more than or equal to x and preferably x and more carbon atoms.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

### DETAILED DESCRIPTION

In the proposed process, C3-C8+ hydrocarbon feed stock is first charged to a “naphtha to ethane and propane” unit to convert naphtha into desirable ethane and propane along with less desirable methane. The produced ethane is fed to an ethylene producing unit. These units provide over 75% yield of ethane to ethylene. The produced propane is fed to a propylene producing unit which provides over 85% yield of propane to propylene. The methane by-product from the naphtha conversion unit and the ethane and propane producing units can be used as a fuel including fuel needed to operate ethylene and propylene producing units which operate at elevated temperatures. Unconverted or under-converted C4+ components in the reactor outlet may be recycled to the reactor inlet for further processing to ethane and propane. Aromatics may also be recovered and further processed.

In the process proposed herein, it is preferred to contact feed streams with catalysts having lower platinum dispersion to have lower methane at given levels of ethane or ethane to propane production when testing under metal-function limited conditions (low hydrogen/hydrocarbon ratios). The low platinum dispersion can be attained by steaming the catalyst after incorporating the platinum onto the catalyst (if platinum was ion exchanged onto zeolite micropores using Pt(2+) precursor). Subjecting the platinum incorporated catalyst to steaming would drive platinum initially located inside the zeolite to the exterior of the zeolite crystal and possibly onto the binder as well. Alternatively, platinum can be introduced onto the binder by using platinum precursors that would selectively chemisorb onto the binder (such as chloroplatinic acid in silica bound zeolite catalyst). Furthermore, by incorporating pertinent competitive ions such as NH₄NO₃ (for Pt (II) precursor) and HCl (for CPA precursor), one can tune the platinum distributions between zeolite micropore and zeolite exteriors/binder to minimize methane production, while maximizing ethane production. When operating the naphtha to ethane and propane reaction under acidity limited conditions (higher H₂/hydrocarbon ratio), it is desired to operate with catalysts having a minimal acidity of 0.10, preferably 0.12 and most preferably 0.15 mmoles/gram acidity as per NH3-TPD measurement. Operating with low acidity catalysts would require high temperatures thereby producing high methane at target ethane production.

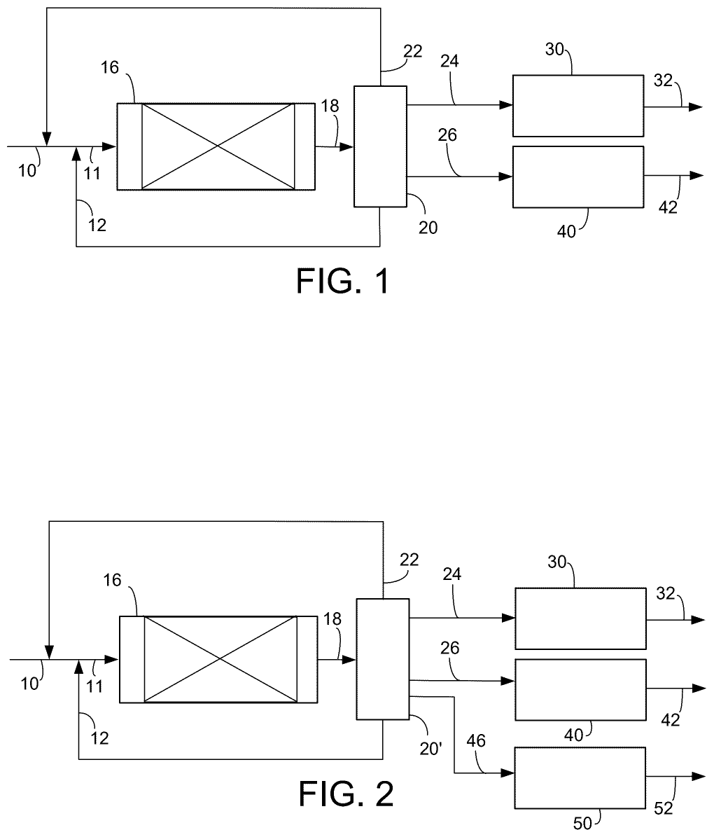

Turning to FIG. 1, a naphtha stream in line 10 may be combined with a hydrogen stream in line 22 and a heavy stream in line 12 to provide a charge stream in line 11, heated and charged to a naphtha to ethane and propane (NEP) reactor 16 to be contacted with an NEP catalyst. The naphtha stream may comprise C4 to C12 hydrocarbons preferably having a T10 between about −10° C. and about 60° C. and a T90 between about 70 and about 180° C. The naphtha feed stream may comprise normal paraffins, iso-paraffins, naphthenes, and aromatics. The naphtha stream may be heated to a reaction temperature of about 300° C. to about 600° C., suitably between about 325° C. and about 550° C., and preferably between about 350° C. and about 525° C. Weight space velocity should be between about 0.3 to about 20 hr−1, suitably between about 0.5 and about 10 hr−1 and preferably between about 1 to about 4 hr−1. A total pressure should be about 0.1 to about 3 MPa (abs), preferably no more than 2 MPa (abs). At these conditions, C2-C4 yield is consistently in an excess of 80 wt %, while methane yield is less than about 10 wt %, suitably below about 8 wt % and typically below about 6 wt % and preferably no more than 5 wt %. Under these conditions, ethane can make up more than 60 wt % of the total C2 to C3 and for that matter C2 to C4 produced in the NEP reactor 16.

The hydrogen-to-hydrocarbon molar ratio is important to producing ethane and propane. The hydrogen-to-hydrocarbon ratio should be about 0.3 to about 15 and preferably about 0.5 to about 5. In a further embodiment, the hydrogen-to-hydrocarbon molar ratio may typically be no more than 5, suitably be no more than 3 and preferably be no more than 2. Low hydrogen-to-hydrocarbon ratio promotes desired reaction kinetics which are initiated with dehydrogenation. Hydrogen-to-hydrocarbon ratio may range from about 50% to about 500%, suitably no more than 300% and preferably no more than 200%, of stoichiometric requirements to convert naphtha molecules to ethane and/or propane.

The molar ratio of hydrogen to hydrocarbon depends on the feed type including paraffin, naphthene or aromatics, the feed molecular carbon number, and the desired product between predominantly ethane, predominantly propane or ethane and propane of comparable abundance as illustrated in Table 1 below. For example, converting 1 mole of propane to ethane at stoichiometry, the process would require co-feeding 0.5 moles of hydrogen. In practice, the process can operate above or below this stoichiometry of 0.5 such as 0.33 to achieve greater than 40% ethane and less than 15% methane, depending on the process design parameters such as, feed contaminants, reactor type (fixed bed, moving bed, fluidized bed), and regeneration frequency. As the carbon number of feed molecules increases from light naphtha (C₅-C₇) to full range naphtha (C₆-C₁₀) the amount of hydrogen required for the reaction increases. For example, it would require 3.5 moles hydrogen and 2.0 moles of hydrogen to fully convert 1 mole of nonane to ethane and propane, respectively. The disclosed process can operate at three to five times the hydrogen-to-hydrocarbon ratio required to stoichiometrically convert the feed molecules to ethane and propane, respectively. It can also operate at 50% of hydrogen-to-hydrocarbon ratio required to stoichiometrically convert the feed molecules to ethane and propane, respectively. The hydrogen-to-hydrocarbon ratio would also depend on the need to produce petrochemical aromatics such as benzene, toluene and xylene. Following these guidelines, one can utilize the disclosed process to convert light naphtha (C₅-C₇), full range naphtha (C₅-C₁₀) and hydrocracking-derived light and heavy naphtha comprising paraffins, naphthenic and aromatic molecules, to ethane, propane, petrochemical aromatics and mixtures thereof.

TABLE 1

| | Desired Reaction | | | |
|---|---|---|---|---|
| | Paraffin to | Paraffin to | Naphthene | Naphthene |
| Carbon | Ethane | Propane | to Ethane | to Propane |
| Number | Stochiometric Hydrogen-to-Hydrocarbon Molar Ratio | | | |
| 3 | 0.5 | | | |
| 4 | 1 | 0.333 | | |
| 5 | 1.5 | 0.667 | 2.5 | 1.667 |
| 6 | 2 | 1.000 | 3.000 | 2.000 |
| 7 | 2.5 | 1.333 | 3.500 | 2.333 |
| 8 | 3 | 1.667 | 4.000 | 2.667 |
| 9 | 3.5 | 2.000 | 4.500 | 3.000 |
| 10 | 4 | 2.333 | 5.000 | 3.333 |

The NEP catalyst dehydrogenates the paraffin molecules in the naphtha to their olefinic analog, interconverts the olefins to lighter olefins and hydrogenates the lighter olefins to produce a light paraffin stream comprising ethane and propane. Interconversion can mean that olefins are also oligomerized to higher olefins and then these higher olefins are cracked to lower olefins. This chemical mechanism avoids or minimizes hydrogenolysis and pyrolytic cracking reactions which produce methane. Methane is an undesirable by-product that represents an opportunity lost for producing valuable ethane and propane and consumes excessive hydrogen. The NEP catalyst can also dehydrogenate naphthenes to aromatics such as benzene, toluene and xylene.

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙAlₙSi₉₆-nO₁₉₂·16H₂O (0<n<10). In various embodiments, the ZSM-5 zeolite may comprise a silica-to-alumina molar ratio of 20 to 1000, 20 to 800, 20 to 600, 25 to 400, 25 to 200 or 25 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicone, zinc, titanium, zirconium and mixtures of thereof. In an embodiment, the binder may comprise a phosphate in the binder or a phosphate of the forenamed oxide binder materials. Preferably, the binder is an aluminum phosphate. The MFI zeolite may be supported in an alumina containing binder such as aluminum phosphate.

MFI zeolite slurry may be first mixed with a binder in the form of colloidal suspension (sol) and gelling reagent and then dropped into hot oil to make spheres controlled to produce 1/64-inch to about 1/10-inch diameter calcined supports. Spheres may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

In an embodiment, the NEP catalyst comprises a metal on the catalyst. The metal may comprise a transition metal. In a further embodiment, the metal may comprise platinum, palladium, iridium, rhenium, ruthenium and mixtures thereof. The metal may be a noble metal. In an additional embodiment, a modifier metal may also be included on the catalyst. The modifier metal may include tin, germanium, gallium, indium, thallium, zinc, silver and mixtures thereof. The modifier metal should be more concentrated on the binder than on the zeolite. In an embodiment, about 0.01 to about 5 wt % of each of the transition metal and the modifier metal are on the catalyst. The catalyst may comprise about 0.1 to about 3 wt % transition metal.

Metal may be incorporated into the binder by evaporative impregnation. A solution of platinum such as tetraamine platinate nitrate or chloroplatinic acid may be contacted with the spheric supports which have been calcined and ion-exchanged in a rotary evaporator, followed by drying and oxidation.

In a preferred embodiment, the NEP catalyst comprises a metal on the spheric supports of the catalyst. Preferably, more of the metal is on the binder than on the zeolite. In an embodiment, at least 60 wt %, suitably at least 70 wt %, preferably at least 80 wt % and most preferably at least 90 wt % of the metal is on the binder. In an embodiment, the zeolite and/or the entire NEP catalyst is steamed oxidized to drive the metal off the zeolite. Steaming is preferably performed after the metal is added to the catalyst. The dried, metal-impregnated binder supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts are preferably reduced to activate them for catalyzing the NEP reaction. For example, the catalyst may be reduced in flowing hydrogen at about 500 to about 550° C. for about 2 to about 5 hours before contacting feed.

After paraffin conversion, a light paraffin stream is discharged from the NEP reactor 16 in an effluent line 18. The light paraffin stream may comprise at least about 40 wt % ethane or at least about 40 wt % propane or at least about 70 wt % and preferably at least about 80 wt % ethane and propane. The ethane to propane ratio can range from about 0.1 to about 5. The light paraffin stream can have less than about 15 wt %, suitably less than about 12 wt %, more suitably less than about 10 wt %, preferably less than about 8 wt %, more preferably less than about 6 wt % and most preferably less than about 5 wt % methane.

We have found that the presence of sulfur in the NEP reaction does not significantly impact conversion. At least about 5 wppm sulfur can be present in the feed without significant impact on conversion. We expect that the NEP catalyst can handle as much as about 100 and perhaps about 200 wppm sulfur without significant impact on conversion.

The light paraffin stream may be cooled and fed to an NEP separation unit 20. The NEP separation unit 20 may be a fractionation column or a series of fractionation columns and other separation units that may separate the light paraffin stream in line 18 into the hydrogen stream in line 22, an ethane stream in line 24, a propane stream in line 26 and the heavy stream in line 12. The NEP separation unit 20 may comprise a demethanizer column that separates the light paraffin stream into a gas stream in an overhead line and a C2+ paraffin stream in a bottoms line. The gas stream may be sent to a hydrogen purification unit such as a PSA unit to recover hydrogen in line 22 for recycle to the NEP reactor 16. Remaining methane from the hydrogen purification unit may be used for fuel gas. The C2+ paraffin stream may then be fed to a deethanizer column to produce the ethane stream in a deethanizer overhead line 24 and a C3+ paraffin stream in a deethanized bottoms line. The C3+ paraffin stream may then be fed to a depropanizer column to produce the propane stream in a depropanizer overhead line 26 and the heavy paraffin stream in the recycle line 12 which may comprise C4+ hydrocarbons. The NEP separation unit 20 may take other forms.

For example, the NEP separation unit 20 may omit a demethanizer column and the light paraffin stream in line 18 may feed a deethanizer column which produces a C2-stream in a deethanizer overhead line. The C2-stream can be separated in the hydrogen purification unit to recover a hydrogen stream in line 22 while residual ethane and methane from the hydrogen purification unit can comprise or supplement the ethane stream in line 24. The hydrogen purification unit may comprise a membrane unit and the hydrogen recovered from the membrane unit may be further purified in an absorption column before it is recycled to the NEP reactor 16 in line 22. In an additional alternative, the C2-stream from the deethanizer column may be charged to an ethylene producing unit 30 in which ethane is converted to ethylene but methane and hydrogen rides through inertly to be recovered in a downstream ethylene recovery unit.

The ethane stream in line 24 may be charged to an ethylene producing unit 30 in which ethane in the ethane stream is converted into ethylene. In an embodiment, the ethylene producing unit 30 is a steam cracking unit. The ethane stream in line 24 may be cracked under steam in a furnace to produce a cracked stream including an ethylene stream 32. The ethane stream may be charged to the ethane steam cracking unit in the gas phase. The ethane steam cracking unit may preferably be operated at a temperature of about 750° C. (1382° F.) to about 950° C. (1742° F.). The cracked stream exiting the furnace of the ethane steam cracking unit may be in a superheated state. One or more quench columns, or other devices known in the art, but preferably an oil quench column and/or a water quench column, may be used for quenching or separating the cracked stream into a plurality of cracked streams. The ethane steam cracking unit may further comprise additional distillation columns, amine wash columns, compressors, expanders, etc. to separate the cracked stream into cracked streams rich in individual light olefins the most predominant of which is the ethylene stream in line 32. The ethylene stream may comprise a yield of at least 75 wt %, preferably at least 80 wt %, ethylene based on the ethane stream in line 24. Among the other components in the cracked stream exiting the ethane steam cracking, ethylene producing unit 30 may be hydrogen, methane, propylene, butene, and pyrolysis gas. Each of these components may be recovered and further processed.

The ethylene stream in line 32 and a propylene stream from the ethylene producing unit 30 may be recovered or transported to polymerization plants, chemical plants or exported. A butene stream may be recovered and used to produce plastics or other petrochemicals by processes such as polymerization or exported. Product recovery of at least 50 wt %, typically at least 60 wt % and suitably at least 70 wt % of valuable ethylene, propylene, and butylene products is achievable from the ethane steam cracking unit 30 based on the ethane stream in line 32.

In another embodiment, the ethylene producing unit 30 may be an oxidative dehydrogenation (ODH) unit. The ethane stream in line 24 may be charged to the ODH unit. An ethane ODH process is an alternative technology to ethane steam cracking or ethane pyrolysis for the conversion of ethane into ethylene. Ethane ODH involves contacting an ethane feed and an oxygen source in the presence of an ODH catalyst in an ODH reaction zone under conditions to oxidatively dehydrogenate at least a portion of the ethane to produce a product stream comprising ethylene, carbon oxides, water, and unreacted oxygen, acetic acid and other organic acids and unconverted ethane. The oxygen source can be an oxygen containing stream or an oxygen containing material such as a metal oxide. Mixed-metal-oxide catalysts have been found to be effective for oxidative dehydrogenation reactions.

The ODH reactor may use a mixed-metal oxide catalyst and operate at temperatures of about 300 to about 400° C. and produce over 90% ethylene and acetic acid, both useful products. The ODH unit produces the ethylene stream in line 32 along with an acetic acid stream. Ethane oxidative dehydrogenation using the mixed-metal oxide catalysts may be carried out at a temperature from about 300° C. to about 500° C., preferably from about 350° C. to about 450° C. at a pressure of from about 0.1 to about 20 barg, preferably from about 0.1 to about 10 barg, a space velocity of from about 1000 to about 5000 cm³/(gcat·hr), wherein the molar ratio of ethane to oxygen is about 1.5:1 to 2:1 with sufficient inert diluent to achieve safe operating conditions. MoVNbTe-oxide catalysts (and other related materials) with the M1-type structure are believed to be the best catalysts for ethane ODH.

Ethane ODH using a mixed-metal oxide catalyst in which the catalyst is also the oxygen source may be carried out at a temperature from about 600° C. to about 900° C., preferably from about 750° C. to about 850° C. at a pressure of from about 0.1 to about 20 barg, preferably from about 0.1 to about 10 barg, and a gas hourly space velocity of from about 1000 to about 5000 h−1. Mg6Mn08-oxide catalysts (and other related materials) have been identified as preferable materials for ethane ODH processes in which the catalyst is also the oxygen source.

The propane stream in line 26 may be charged to a propylene producing unit 40 in which propane in the propane stream is converted into propylene. The propylene producing unit 40 may be a paraffin dehydrogenation (PDH) unit. PDH catalyst is used in a dehydrogenation reaction process to catalyze the dehydrogenation of paraffins, such as propane. The conditions in the dehydrogenation reactor may include a temperature of about 500 to about 800° C., a pressure of about 40 to about 310 kPa and a catalyst to oil ratio of about 5 to about 100.

The dehydrogenation reaction may be conducted in a fluidized manner such that gas, which may comprise the reactant paraffins with or without a fluidizing inert gas, is distributed to the reactor in a way that lifts the dehydrogenation catalyst in the reactor vessel while catalyzing the dehydrogenation of paraffins. During the catalytic dehydrogenation reaction, coke is deposited on the dehydrogenation catalyst leading to reduction of the activity of the catalyst. The dehydrogenation catalyst must then be regenerated in a regenerator. The regenerator may combust coke from the dehydrogenation catalyst and fuel gas to ensure sufficient enthalpy in the dehydrogenation reactor to promote the endothermic reaction.

The dehydrogenation catalyst selected should minimize cracking reactions and favor dehydrogenation reactions. Suitable catalysts for use herein include an active metal which may be dispersed in a porous inorganic carrier material such as silica, alumina, silica alumina, zirconia, or clay. An exemplary embodiment of a catalyst includes alumina or silica-alumina containing gallium, a noble metal, and an alkali or alkaline earth metal.

The catalyst support comprises a carrier material, a binder and an optional filler material to provide physical strength and integrity. The carrier material may include alumina or silica-alumina. Silica sol or alumina sol may be used as the binder. The alumina or silica-alumina generally contains alumina of gamma, theta and/or delta phases. The catalyst support particles may have a nominal diameter of about 400 to about 5000 micrometers with the average diameter of about 600 to about 3500 micrometers. Preferably, the surface area of the catalyst support is about 85 to about 140 m2/g.

The fluidized dehydrogenation catalyst may comprise a dehydrogenation metal on a support. The dehydrogenation metal may be a one or a combination of transition metals. A noble metal may be a preferred dehydrogenation metal such as platinum or palladium. Gallium is an effective metal for paraffin dehydrogenation. Metals may be deposited on the catalyst support by impregnation or other suitable methods or included in the carrier material or binder during catalyst preparation.

The acid function of the catalyst should be minimized to prevent cracking and favor dehydrogenation. Alkali metals and alkaline earth metals may also be included in the catalyst to attenuate the acidity of the catalyst. Rare earth metals may be included in the catalyst to control the activity of the catalyst. Concentrations of 0.001% to 10 wt % metals may be incorporated into the dehydrogenation catalyst. In the case of the noble metals, it is preferred to use about 10 parts per million (ppm) by weight to about 600 ppm by weight noble metal. More preferably it is preferred to use about 10 to about 100 ppm by weight noble metal. The preferred noble metal is platinum. Gallium should be present in the range of 0.3 wt % to about 3 wt %, preferably about 0.5 wt % to about 2 wt %. Alkali and alkaline earth metals may be present in the range of about 0.05 wt % to about 1 wt %.

Regenerated catalyst may be contacted with the propane stream in line 26 perhaps with a fluidizing gas to lift the propane stream and dehydrogenation catalyst up a riser while dehydrogenation occurs. Above the riser spent dehydrogenation catalyst and propylene product may be separated by a centripetal separation device. Propylene product gas may be quenched with a cooling fluid to prevent over reaction to undesired by-products. Separation of the propylene product may include quench contacting and fractionation to produce a propylene product stream in line 42. Unreacted propane may be recycled to the dehydrogenation reactor and lighter gases may be recycled to the regenerator as fuel gas to be combusted to provide enthalpy for the reaction.

The propylene producing unit may also employ a catalytic moving bed reactor. The reactor section may comprise several radial flow reactors in parallel or series heated by charge and interstage heaters. The propane stream perhaps with added hydrogen flows in each dehydrogenation reactor from a screened center pipe through an annular dehydrogenation catalyst bed to an outer effluent annulus. Flow may be in the reverse fashion. The dehydrogenation catalyst may comprise a noble metal or mixtures thereof, a modifier selected from the group consisting of alkali metals or alkaline-earth metals and mixtures thereof, a component selected from the group consisting of tin, germanium, lead, indium, gallium, thallium, and mixtures thereof, and a porous support forming a catalyst particle. The catalyst support may comprise oil dropped alumina spheres.

Dehydrogenation conditions may include a temperature of from about 400 to about 900° C., a pressure of from about 0.01 to 10 atmospheres absolute, and a liquid hourly space velocity (LHSV) of from about 0.1 to 100 hr−1. The pressure in the dehydrogenation reactor is maintained as low as practicable, consistent with equipment limitations, to maximize chemical equilibrium advantages. Spent dehydrogenation catalyst in the annular catalyst bed may be withdrawn from the bottom of the bed, forwarded to a regenerator to combust coke from the catalyst with air at about 450 to about 600° C. Noble metal on the catalyst may be redispersed by an oxyhalogenation process, dried and returned to the top of the dehydrogenation catalyst bed as regenerated dehydrogenation catalyst.

Dehydrogenation effluent from the propylene producing unit 40 may be cooled, compressed, dried and hydrogen is cryogenically separated from the hydrocarbons with a net gas purity of 85 to 93 mol % hydrogen. Hydrocarbon liquid is selectively hydrogenated to convert diolefins and acetylenes and the hydrocarbon liquid is fractionated in a deethanizer column to remove ethane and propylene is split from propane in a propane-propylene splitter column to provide polymer-grade propylene in line 42. Propane may be recycled as feed to the propylene producing unit 40.

The heavy stream in line 12 which may be taken from a bottom of a depropanizer column may comprise C4+ paraffins. The heavy stream in line 12 may be recycled to the NEP reactor 16 by combination with the paraffin stream in line 10 and the hydrogen stream in line 22 and charged to the NEP reactor in line 11 to produce more ethane and propane.

In an alternative embodiment, the light paraffin stream in line 18 may be separated by the NEP separation unit 20 into a hydrogen stream in line 22, an ethane stream in line 24 and a heavy stream in line 12 as previously described, but the propane stream in line 26 can include isobutane. In this embodiment, a propane and isobutane stream in line 26 can be fed to the propylene producing unit 40. The propylene producing unit 40 can be equipped to dehydrogenate propane in the propane and isobutane stream 26 to propylene and isobutane in the propane and isobutane stream in line 26 to isobutylene in the same dehydrogenation reactor(s). The fractionation section from the propylene producing unit 40 may include a depropanizer column downstream of the bottoms line of the propylene-propane splitter column to separate unreacted propane in an overhead from C4 hydrocarbons. An isobutylene-isobutane splitter column in downstream communication with a bottom of the depropanizer column may provide isobutylene in the overhead that can be recovered as product and unreacted isobutane in the splitter bottoms may be returned to the propylene producing unit 40 to be converted to isobutylene.

Unreacted normal C4+ hydrocarbons comprising normal C4 and higher paraffins in the heavy stream 12 may be recycled to be combined with the naphtha stream in the feed line 10 and the hydrogen stream in line 22 and charged to the NEP reactor 16 in line 11.

FIG. 2 shows an embodiment of an alternative embodiment to the embodiment of FIG. 1 which employs a normal butylene conversion unit 50. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is essentially the same as in FIG. 1 with the following exceptions.

The NEP separation unit 20′ additionally provides a normal butane product stream in line 46 in addition to the hydrogen stream in line 22, the ethane stream in line 24 and the propane and isobutane stream in line 26. The normal butane stream in line 46 may be charged to a butylene producing unit 50 for conversion of the normal butane in the normal butane stream to normal butylene. This embodiment may be useful in cases in which the propylene producing unit cannot process propane and normal butane in the same dehydrogenation reactor. The butylene producing unit 50 may include a dehydrogenation unit as described for the propylene producing unit in the embodiment of FIG. 1.

The butylene producing unit 50 may feed a normal butylene stream to a normal butylene-normal butane splitter column. A normal butylene stream in a splitter overhead line may be taken as normal butylene product in line 52 while the unreacted normal butane in the bottoms line may be recycled back to the dehydrogenation unit in the butylene producing unit 50 to be converted to normal butylene.

In some cases, the butylene producing unit 50 may be a butylene producing unit that is able to convert both normal butane and isobutane to normal butene and isobutene. In such a case, the line 46 will carry a butane stream which is lean of propane to a butylene producing unit 50 and the propane stream in line 26 will carry a propane stream that is lean in butane to the propylene producing unit. The butylene producing unit will produce a butene product stream in line 52 that may comprise isobutene and butene.

The heavy stream in line 12 from the NEP separation unit 20′ may comprise a C5+ hydrocarbon stream although it may include C4 hydrocarbons and comprise a C4+ hydrocarbon stream. Unreacted C4+ or C5+ hydrocarbons comprising C4 or C5 and heavier hydrocarbons in the heavy stream 12 may be recycled to be combined with the naphtha stream in the feed line 10 and the hydrogen stream in line 22 and charged to the NEP reactor 16 in line 11.

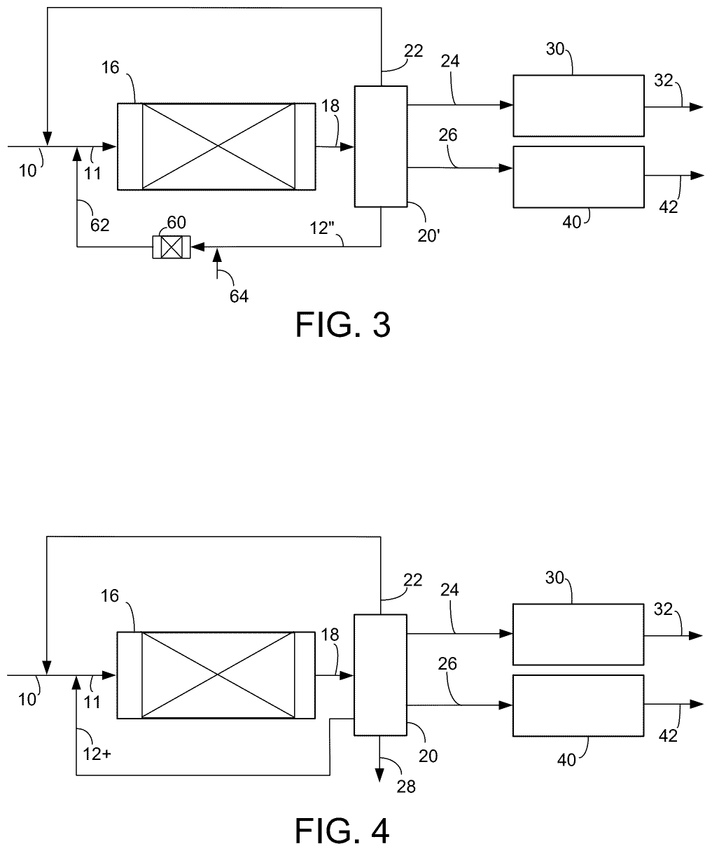

We have also found that the heavy paraffin stream in line 12 may also comprise aromatics, benzene, toluene and xylene. FIG. 3 shows an embodiment of an alternative embodiment to the embodiment of FIG. 1 which maximizes ethane and propane production by hydrotreating the entire heavy stream in line 12″ in a hydrotreating reactor 60 to saturate aromatic rings to naphthenes providing a recycle feed in line 62 to the NEP reactor 16. Elements in FIG. 3 with the same configuration as in FIG. 1 will have the same reference numeral as in FIG. 1. Elements in FIG. 3 which have a different configuration as the corresponding element in FIG. 1 will have the same reference numeral but designated with a double prime symbol (″). The configuration and operation of the embodiment of FIG. 3 is essentially the same as in FIG. 1 with the following exceptions.

The heavy stream in line 12″ comprising C4+ paraffins and aromatics, benzene, toluene and xylene is mixed with a hydrogen stream in line 64, heated and charged to the hydrotreating reactor 60.

The hydrotreating reactor 60 may have one or more beds of hydrotreating catalyst to saturate aromatic rings in the heavy stream. The heavy stream may be charged to the hydrotreating reactor 60 at a hydrotreating inlet temperature that may range from about 200° C. (392° F.) to about 400° C. (752° F.). The hydrotreating reactor 60 may employ interbed hydrogen quench streams if more than one catalyst bed is used.

Suitable hydrotreating catalysts are any known conventional hydrotreating catalysts and include those which are comprised of at least one Group VIII metal, preferably iron, cobalt and nickel, more preferably cobalt and/or nickel and at least one Group VI metal, preferably molybdenum and tungsten, on a high surface area support material, preferably alumina. Other suitable hydrotreating catalysts include zeolitic catalysts, as well as noble metal catalysts where the noble metal is selected from palladium and platinum. It is within the scope of the present description that more than one type of hydrotreating catalyst be used in the same hydrotreating reactor 60. The Group VIII metal is typically present in an amount ranging from about 2 to about 20 wt %, preferably from about 4 to about 12 wt %. The Group VI metal will typically be present in an amount ranging from about 1 to about 25 wt %, preferably from about 2 to about 25 wt %. Generally, hydrotreating conditions include a pressure of about 700 kPa (100 psig) to about 21 MPa (3000 psig). The hydrotreating outlet temperature may range between about 300° C. (572° F.) and about 427° C. (800° F.).

The saturated aromatics and C4+ paraffins in a hydrotreated heavy stream in line 62 may be recycled to join the naphtha stream in line 10 and the hydrogen stream in line 22 and be charged to the NEP reactor 16 in line 11. Hydrogen from the hydrotreating reactor 60 may be recycled with the saturated aromatics to the NEP reactor 16 to reduce or eliminate hydrogen requirements from line 22.

In another embodiment, the NEP separation unit 20+ includes a debutanizer column that separates C4 and perhaps C5 hydrocarbons for recycle to the NEP reactor 16 in line 12+ while preserving aromatics for further processing and valorization. FIG. 4 shows an alternative embodiment to the embodiment of FIG. 1 which preserves aromatics. Elements in FIG. 4 with the same configuration as in FIG. 1 will have the same reference numeral as in FIG. 1. Elements in FIG. 4 which have a different configuration as the corresponding element in FIG. 1 will have the same reference numeral but designated with a cross symbol (+). The embodiment of FIG. 4 operates and is configured essentially as FIG. 1 with the following exceptions.

The NEP separation unit 20+ includes a debutanizer column that separates a heavy stream comprising C4 and perhaps C5 paraffins from a depropanizer bottoms stream in a debutanizer overhead line for recycle to the NEP reactor 16 in line 12+ and C5+ or C6+ aromatics in a debutanized bottoms line 28. The aromatics in the debutanized bottoms line 28 may be further processed for valorization of the valuable aromatics.

The foregoing disclosure provides a process for converting naphtha to ethane and propane feed with maximized conversion to ethylene and propylene in ethylene and propylene producing units, respectively. An increased amount of ethane is produced with the process provided here and therefore a higher amount of ethylene can be produced.

### Examples 1 and 2

Zeolite MFI with a Si/Al₂ ratio of 40 was synthesized by crystallizing a mixture consisting of silica, alumina, alkali metal and an OSDA comprising tetrapropyl ammonium (TPA). As synthesized zeolite MFI was first mixed with AlPO₄ sol and gelling reagent, and the formed slurry was dropped into hot oil. The size of the spheres was controlled to attain 1/16-inch diameter calcined support. The formed spheres coming off sphere forming section was separated from the oil and transported to the washing section for ammonia wash to remove sodium ions off the zeolite. The wet spheres were dried and then calcined to remove the OSDA. Optionally the calcined support is NH₄-ion exchanged using an ammonium nitrate solution to remove residual sodium ions, followed by a drying step at about 110° C. prior to a metal incorporation step. Platinum incorporation was performed by an evaporative impregnation technique via contacting the supports with a solution of platinum containing compounds such as tetraamine platinate nitrate in a rotary evaporator, followed by drying with heat introduced from a steam jacket. Once the spheres become free rolling, the impregnated support was steam oxidized at 607° C. with 7.7 or 14.3 mol % steam in flowing air for 2 hours in a quartz tube in a 3-zone furnace. The levels of platinum on the finished catalysts were targeted at 0.005, 0.02, 0.10 and 0.40 wt % and are designated as Comparative Examples and Examples 1.1 through 1.4 (for 7.7% steam) and 2.1 through 2.4 (for 14.3% steam) as shown in Table 2.

TABLE 2

| | | | | | | | | | high |
|---|---|---|---|---|---|---|---|---|---|
| | | | | | | | | | temp |
| | MFI | | | | steam | | | H2 | acidity, |
| | Si/ | | Pt | % | temp, | wt % | H2/ | umol/ | mmol/ |
| | Al2 | binder | precursor | H2O | ° C. | Pt | Pt | gm | gm |
| Comp Ex. 1.1 | 40 | AlPO4 | TAPN | 7.7 | 607 | 0.005 | | | |
| Ex. 1.2 | 40 | AlPO4 | TAPN | 7.7 | 607 | 0.02 | | | |
| Ex. 1.3 | 40 | AlPO4 | TAPN | 7.7 | 607 | 0.1 | 0.08 | 0.43 | 0.31 |
| Ex. 1.4 | 40 | AlPO4 | TAPN | 7.7 | 607 | 0.4 | 0.07 | 1.44 | 0.32 |
| Comp Ex. 2.1 | 40 | AlPO4 | TAPN | 14.4 | 607 | 0.005 | | | |
| Ex. 2.2 | 40 | AlPO4 | TAPN | 14.4 | 607 | 0.020 | | | |
| Ex. 2.3 | 40 | AlPO4 | TAPN | 14.4 | 607 | 0.100 | 0.04 | 0.22 | 0.28 |
| Ex. 2.4 | 40 | AlPO4 | TAPN | 14.4 | 607 | 0.400 | 0.04 | 0.84 | 0.23 |

### Example 3

A series of extrudates of ⅛-inch trilobed shape were prepared with a formulation of 65% MFI-40 prepared according to that in Example 1 and 35% of Hisil 532 SiO₂ and Ludox SiO₂ combined. An extrusion aid of 1% Methocel A was incorporated into the formulation. The extrudate was dried at 110° C., calcined at 520° C. over a flowing air in a muffle oven. The calcined support was ammonium ion exchange using 1.0 M of ammonium nitrate solution to remove residual sodium. The ammonium ion exchange support was dried at 110° C. and chloroplatinic acid (CPA) was subsequently incorporated onto the support by evaporative impregnating technique to attain 0.2% platinum on the finished catalysts. The impregnated support was steam oxidized at a temperature range of 565 to 650° C. and over a steam range of 7.7 to 20% for 2 hours. The finished catalyst is designated as Comparative Examples and Examples 3.1 through 3.11 as shown in Table 3. The accessible platinum (or platinum dispersion) was determined by hydrogen chemisorption by volumetric vacuum method prescribed essentially according to ASTM D3908-20. The available chemisorption sites are reported as umoles H₂ per gram catalyst and platinum dispersion is calculated using the amount of chemisorption H₂ and platinum contents of the catalysts. The acidity of catalysts was determined by ammonia temperature programming desorption (NH₃-TPD) or calculated based on the correlations between NH₃-TPD and by zeolite framework compositions measured by infrared (stretching frequency) technique. Acidity measurement by NH₃-TPD was conducted using Micromeritics AutoChem 2920 equipped with TCD. In this measurement of acidity, about 250-600 mg are loaded and pretreated by ramping the temperatures at 10° C./min from 25 to 650° C. in 100 ml of 20/80 O₂/helium gas flow and hold at 650° C. for 1 hour. Once cooling down to 150° C. NH₃ adsorption was performed at 150° C. flow by exposing the sample to NH₃, which is done by sweeping several loops (volume per loop) of 10/90 NH₃/helium gas mixture using 20 ml/min helium flow into the sample chamber. Thereafter, a 75 ml/min of helium flow is passing through the sample chamber to purge off NH₃ not or loosely adsorbed onto the sample at 150° C. until no effluent composition changes. Following NH₃ purge, NH₃ desorption is performed by ramping the sample chamber temperatures from 150 to 650° C. at 10° C./min with a 75 ml/min helium flow. NH₃ coming off the sample is monitored using TCD and results are reported in micromoles of NH₃ desorbed over a specified range of temperatures per gram. Acidity by NH₃-TPD is characterized by a low temperature peak around 250° C. and a high temperature peak around 400° C. and the acidity pertaining to the high temperature peaks are reported in tables. When NH₃-TPD results are missing, NH₃-TPD acidity is derived based on its correlations with framework compositions (acidity) derived from infrared spectroscopy as described below.

The framework compositions (SiO₂ and Al₂O₃) by infrared technique are conducted as per procedure described herein. First, the samples were ground into fine powder using an agate mortar and pestle. The powder was mixed with XL Ultra Pure Spectrograde KBr powder to obtain a dilution of about 1000:1. 13 mm diameter pellets were made by pressing 250 mg of the sample/KBr mixture at 10,000 lbs using a hydraulic press. KBr pellets were dried at 160° C. in a drying oven for >2 hrs. Utilizing a Nicolet is −50 FTIR Spectrometer with DTGS detector, spectra were collected in transmission mode at 2 cm⁻¹ resolution with 128 scans. A standard beta zeolite was used as a daily reference material. The zeolitic asymmetric T-O-T (T=Si, Al) band position is inversely related to framework Al content in many zeolites.

TABLE 3

| | | | | | | | High |
|---|---|---|---|---|---|---|---|
| | | Steam | | | H2 | Calc total | temp |
| | % | temp', | wt % | H2/ | umo/ | acidity, | acidity, |
| Catalyst s | H2O | ° C. | Pt | Pt | gm | mmol/gm | mmol/gm |
| Ex. 3.1 | 7.7 | 575 | 0.2 | 0.17 | 1.74 | 0.421 | 0.274 |
| Ex. 3.2 | 7.7 | 575 | 0.2 | 0.17 | 1.74 | 0.421 | 0.274 |
| Comp 3.3 | 20 | 607 | 0.2 | 0.11 | 1.13 | 0.201 | 0.100 |
| Ex. 3.4 | 10 | 575 | 0.2 | 0.08 | 0.82 | 0.402 | 0.259 |
| Ex. 3.5 | 7.7 | 575 | 0.2 | 0.03 | 0.31 | 0.431 | 0.282 |
| Ex. 3.6 | 7.7 | 575 | 0.2 | 0.07 | 0.72 | 0.368 | 0.232 |
| Ex. 3.7 | 14.3 | 607 | 0.2 | 0.1 | 1.03 | | |
| Ex. 3.8 | 14.3 | 607 | 0.2 | 0.09 | 0.92 | 0.268 | 0.153 |
| Comp 3.9 | 14.4 | 650 | 0.2 | 0.1 | 1.03 | 0.225 | 0.119 |
| Ex. 3.10 | 14.3 | 607 | 0.2 | 0.09 | 0.92 | 0.268 | 0.153 |
| Ex. 3.11 | 14.3 | 607 | 0.2 | 0.09 | 0.92 | 0.268 | 0.153 |

### Example 4

An extrudate of 1/16-inch cylindrical shape was prepared with a formulation of 65% MFI-40 prepared according to that in Example 1 and 35% Hisil 532 SiO₂ and Ludox AS-40 SiO₂ combined. An extrusion aid of 1% Methocel A was incorporated into the formulation. The extrudate was dried at 110° C., calcined at 520° C. over a flowing air in a muffle oven. The calcined support was ammonium ion exchange using 1.0 M of ammonium nitrate solution to remove residual sodium. The ammonium ion exchange support was dried at 110° C. and tetraamine platinate nitrate or chloroplatinic acid was subsequently incorporated onto the support by evaporative impregnating technique to attain either 0.2 or 0.4% platinum on the finished catalysts. The impregnated support was steam oxidized at 607° C. and 14.3% steam for 2 hours and the finished catalysts are designated as Examples 4.1, 4.2 and 4.3, respectively.

Comparative example 4.4 preparation. An alternative preparative sequence to Example 2.4 was employed by first calcining AlPO₄-bound oil dropped spheres, which were ammonium-ion exchanged to remove residual sodium and then steam calcined using 14.3 mol % steam at 607° C. for 2 hours. The steam calcined supports were evaporatively impregnated with a solution of tetraamine platinate nitrate followed by dry oxidation at 520° C. for 2 hours in flowing air. The catalyst contained 0.4 wt % platinum and is designated as Comparative Example 4.4. Comparative Example 4.5 was prepared following the same procedure as Example 4.2 with the exception that the steam oxidation was replaced by dry oxidation at 520° C. Examples 4.6 and 4.7 were prepared following the procedure described for the preparations of Examples 4.3 and 4.2 with the exception MFI-40 was replaced by MFI-23 obtained from Zeolyst. Comparative Example 4.8 was prepared by first preparing silica bound MFI-23 support using oil dropping spheres. The oil dropping support was worked up by calcining the dry base, ion exchanging using ammonium nitrate solution and drying down at 110° C. in a muffle oven. The ammonium ion exchanged support was impregnated with tetramine platinate (II) nitrate to attain a target of 0.2% platinum followed by steam oxidation at 607° C. using a flowing air containing 14.4% H₂O. The support used for Example 4.9 was prepared following the procedures described for the support of Examples 4.1-4.3 but has a trilobed shape of ⅛″ circumference. The platinum incorporation and subsequent work-up follows the same procedure for those of Example 4.1 using a steam oxidation condition of 7.7% steam at 565° C. for 2 hours. Examples 4.10 was obtained by first preparing platinum (0.36% wt) containing particles via impregnating alumina phosphate particle using a chloroplatinic solution, which is then combined with MFI-40 particles followed by pelletized (to 20×60 mesh) and steam oxidation at 7.7% steam and 565° C. for 2 hours. Example 4.11 was obtained by first preparing platinum (0.36% wt) containing particles via impregnating MFI-40 particle using a chloroplatinic solution, which is then combined with alumina phosphate particles followed by pelletized (to 20×60 mesh) and steam oxidation at 7.7% steam and 565° C. for 2 hours. Examples 4.12 was obtained by first preparing platinum (0.36% wt) containing particles via impregnating SiO₂ particle using a chloroplatinic solution, which is then combined with MFI-40 particles followed by pelletized (to 20×60 mesh) and steam oxidation at 7.7% steam and 565° C. for 2 hours.

Descriptions and properties of catalyst Examples and Comparative Examples are summarized in Table 4.

TABLE 4

| | | | | | | | | | high |
|---|---|---|---|---|---|---|---|---|---|
| | | | | | steam | | | | temp |
| | MFI | | Pt | % | temp, | wt % | | H2 | acidity, |
| | Si/Al2 | binder | precursor | H2O | ° C. | Pt | H2/Pt | umol/gm | mmol/gm |
| Ex. 4.1 | 40 | SiO2 | CPA | 14.4 | 607 | 0.20 | 0.13 | 1.35 | 0.16 |
| Ex. 4.2 | 40 | SiO2 | TAPN | 14.4 | 607 | 0.40 | 0.13 | 2.65 | |
| Ex. 4.3 | 40 | SiO2 | TAPN | 14.4 | 607 | 0.20 | 0.31 | 3.22 | |
| Comp. 4.4 | 40 | AlPO4 | TAPN | 0.0 | 520 | 0.40 | 0.35 | 7.22 | 0.30 |
| Comp. 4.5 | 40 | SiO2 | TAPN | 0.0 | 520 | 0.40 | 0.54 | 5.51 | 0.35 |
| Ex. 4.6 | 23 | SiO2 | TAPN | 14.4 | 607 | 0.20 | 0.20 | 2.03 | 0.31 |
| Ex. 4.7 | 23 | SiO2 | TAPN | 14.4 | 607 | 0.40 | 0.17 | 3.57 | |
| Comp. 4.8 | 23 | SiO2 | TAPN | 14.4 | 607 | 0.20 | 0.55 | 5.68 | 0.26 |
| Ex. 4.9 | 40 | SiO2 | CPA | 7.7 | 565 | 0.2 | 0.11 | 1.11 | 0.31 |
| Ex. 4.10 | 40 | AlPO4 | CPA | 7.7 | 565 | 0.127 | 0.06 | 0.38 | |
| Ex. 4.11 | 40 | AlPO4 | CPA | 7.7 | 565 | 0.236 | 0.006 | 0.07 | |
| Ex. 4.12 | 40 | SiO2 | CPA | 7.7 | 565 | 0.127 | 0.19 | 1.22 | |

### Example 5

Catalytic tests were performed in plug flow tubular reactors using an apparatus made up of multiple, parallel reactors. The composition of the feed blend is shown in Table 5, mimicking that of light naphtha. The finished catalyst was crushed and 1 ml of catalyst at 20×60 mesh was loaded in the reactor. The catalyst was pre-reduced at 520° C. for 3 hours in a flowing hydrogen stream, and then lowered to the target reaction temperature, before the feed was introduced. Catalytic performances were measured at about 6 bars pressure, a hydrogen-to-hydrocarbon molar ratio of 2.0, a weight hour space velocity of 2.0 hr−1 over a temperature range of about 390 to about 500° C. The results at 470° C. are shown in Table 6. The results showed a minimal amount of 0.02 wt %, preferably 0.1 wt % and most preferably 0.2 wt % platinum would be required to produce ethane, propane and combination of ethane and propane.

TABLE 5
Simulated Commercial Light Naphtha

| Components | n-C5 | i-C5 | n-C6 | i-C6 | CH & mCP | n-C7 | Sum |
|---|---|---|---|---|---|---|---|
| wt % | 23.09 | 11.54 | 26.40 | 21.10 | 15.82 | 2.05 | 100.00 |

TABLE 6.1

| | Comp Ex | | | |
|---|---|---|---|---|
| Catalysts | 1.1 | Ex. 1.2 | Ex. 1.3 | Ex. 1.4 |
| wt % Pt | 0.005 | 0.02 | 0.1 | 0.4 |
| WHSV_NOM | 2 | 2 | 2 | 2 |
| H2/LN molar | 2.0 | 2.0 | 2.0 | 2.0 |
| temp., C. | 468.2 | 469.5 | 468.4 | 470.6 |
| TOS | 73.2 | 72.1 | 73.6 | 72.5 |
| RXOUT_P, bars | 5.5 | 5.9 | 5.7 | 5.7 |
| C1 | 6.30 | 7.00 | 7.12 | 6.35 |
| C2s | 42.26 | 54.06 | 59.06 | 62.75 |
| C3s | 36.90 | 27.59 | 23.33 | 17.89 |
| C2s + C3s | 79.16 | 81.65 | 82.38 | 80.65 |
| C4s | 3.80 | 3.11 | 2.87 | 2.39 |
| C5s | 0.00 | 0.00 | 0.00 | 0.00 |
| C2-C5 olefins | 0.84 | 0.89 | 0.96 | 1.03 |
| C6s | 0.20 | 0.17 | 0.16 | 0.13 |
| BTX | 9.17 | 6.97 | 6.48 | 8.62 |
| A9 | 0.33 | 0.20 | 0.17 | 0.21 |
| A10 | 0.00 | 0.00 | 0.00 | 0.00 |
| heavy arom | 1.04 | 0.90 | 0.81 | 1.65 |
| sum | 100.00 | 100.00 | 100.00 | 100.00 |

TABLE 6.2

| | Comp Ex | | | |
|---|---|---|---|---|
| Catalyst | 2.1 | Ex. 2.2 | Ex. 2.3 | Ex. 2.4 |
| wt % Pt | 0.005 | 0.02 | 0.1 | 0.4 |
| WHSV_NOM | 2 | 2 | 2 | 2 |
| H2/LN molar | 2.0 | 2.0 | 2.0 | 2.0 |
| temp., C. | 468.2 | 469.5 | 468.4 | 470.6 |
| TOS | 144.0 | 144.0 | 143.0 | 148.0 |
| RXOUT_P, bars | 5.5 | 5.9 | 5.7 | 5.7 |
| C1 | 4.53 | 5.54 | 5.21 | 6.23 |
| C2s | 35.06 | 50.44 | 55.53 | 61.36 |
| C3s | 43.63 | 32.09 | 25.53 | 20.58 |
| C2s + C3s | 78.69 | 82.53 | 81.06 | 81.93 |
| C4s | 4.96 | 4.05 | 3.65 | 2.83 |
| C5s | 0.00 | 0.00 | 0.00 | 0.00 |
| C2-C5 olefins | 0.86 | 1.07 | 1.32 | 1.27 |
| BTX | 10.12 | 6.87 | 8.66 | 7.79 |
| A9 | 0.46 | 0.18 | 0.27 | 0.18 |
| A10 | 0.00 | 0.00 | 0.00 | 0.00 |
| heavy arom | 0.91 | 0.56 | 0.91 | 0.88 |
| sum | 99.67 | 99.74 | 99.75 | 99.84 |

### Example 6

Catalytic tests were performed upon catalysts in Examples 3 in plug flow tubular reactors of ⅞″ diameter. Catalysts of 1/16″ or ⅛″ diameter extrudates were pre-mixed with alpha alumina, before loaded into the reactor. The feed blend is made up of 30% simulated light naphtha as shown in Table 5 and 70% aromatics made up predominantly of toluene. The catalyst was pre-reduced at 520° C. for 3 hours in a flowing hydrogen stream, and then lowered to the target reaction temperature, before the feed was introduced. Catalytic performances were measured at about 21 bars pressure, a hydrogen-to-hydrocarbon (including light naphtha and aromatics) molar ratio of 1.0 over a temperature range of about 400 to about 500° C. (set temperatures). The results at 475° C. are shown in Table 7. It is evident that to produce ethane and propane combined of greater than about 80% and ethane to propane ratios of greater than about 0.5, a minimal acidity of greater than about 0.12 and preferably greater than 0.15 mmoles/gm would be required.

TABLE 7

| | | | | | | | calc high |
|---|---|---|---|---|---|---|---|
| | | steam | wt | C2/ | C2 + | | temp |
| | % | temp', | % | C3, | C3, | CH4 | acidity, |
| catalysts | H2O | ° C. | Pt | w/w | % | % | mmol/gm |
| Ex. 3.1 | 7.7 | 575 | 0.2 | 6.82 | 78.59 | 18.20 | 0.274 |
| Ex. 3.2 | 7.7 | 575 | 0.2 | 7.40 | 79.60 | 18.70 | 0.274 |
| Comp 3.3 | 20 | 607 | 0.2 | 0.31 | 74.91 | 2.40 | 0.100 |
| Ex. 3.4 | 10 | 575 | 0.2 | 3.79 | 82.64 | 14.90 | 0.259 |
| Ex. 3.5 | 7.7 | 575 | 0.2 | 8.1 | 80.17 | 17.45 | 0.282 |
| Ex. 3.6 | 7.7 | 575 | 0.2 | 5.3 | 81.9 | 17.33 | 0.232 |
| Ex. 3.7 | 14.3 | 607 | 0.2 | 2.6 | | | |
| Ex. 3.8 | 14.3 | 607 | 0.2 | 1.8 | 81.02 | 13.63 | 0.153 |
| Comp 3.9 | 14.4 | 650 | 0.2 | 0.5 | 79.1 | 6.36 | 0.119 |
| Ex. 3.10 | 14.3 | 607 | 0.2 | 2.6 | 80.3 | 14.18 | 0.153 |
| Ex. 3.11 | 14.3 | 607 | 0.2 | 2.6 | 83.9 | 13.46 | 0.153 |

### Example 7

Catalytic performances of catalyst Examples 4 were measured using the apparatus and test protocol described in Example 5. The test results were reported as the amount of methane formation at about 55% ethane yields as shown in Table 8. Also included are performances of selective catalyst Examples 1 and 2. The results show methane formation at given ethane production is reduced with lowered amounts of hydrogen chemisorption and lowered chemisorption hydrogen to platinum ratios as shown in Table 8. It is desired to have H₂ chemisorption less than about 7, preferably less than 6 and most preferably less than about 5 umol/gm depending on the binder employed to produce the catalyst. It is desired to have chemisorption hydrogen to platinum ratios of less than about 0.5. preferably less than 0.45 and most preferably less than 0.35.

TABLE 8

| | | | | | steam | Pt | Chemisorb | H2 | | |
|---|---|---|---|---|---|---|---|---|---|---|
| | MFI | | Pt | % | temp, | wt | H2 to Pt | Chemisorb | C2 | CH4 |
| | Si/Al2 | binder | precursor | H2O | ° C. | % | ratios | umol/gm | % | % |
| Ex. 4.1 | 40 | SiO2 | CPA | 14.4 | 607 | 0.20 | 0.13 | 1.35 | 55.0 | 5.6 |
| Ex. 4.2 | 40 | SiO2 | TAPN | 14.4 | 607 | 0.40 | 0.13 | 2.65 | 54.4 | 7.2 |
| Ex. 4.3 | 40 | SiO2 | TAPN | 14.4 | 607 | 0.20 | 0.31 | 3.22 | 54.8 | 7.9 |
| Comp. 4.4 | 40 | AlPO4 | TAPN | 0.0 | 520 | 0.40 | 0.35 | 7.22 | 54.5 | 8.5 |
| Comp. 4.5 | 40 | SiO2 | TAPN | 0.0 | 520 | 0.40 | 0.54 | 5.51 | 54.9 | 13.0 |
| Ex. 4.6 | 23 | SiO2 | TAPN | 14.4 | 607 | 0.20 | 0.20 | 2.03 | 55.0 | 8.2 |
| Ex. 4.7 | 23 | SiO2 | TAPN | 14.4 | 607 | 0.40 | 0.17 | 3.57 | 55.0 | 9.0 |
| Comp. 4.8 | 23 | SiO2 | TAPN | 14.4 | 607 | 0.20 | 0.55 | 5.68 | 55.0 | 13.5 |
| Ex. 4.9 | 40 | SiO2 | CPA | 7.7 | 565 | 0.2 | 0.11 | 1.11 | 55.0 | 4.7 |
| Ex. 4.10 | 40 | AlPO4 | CPA | 7.7 | 565 | 0.127 | 0.06 | 0.38 | 55.0 | 4.7 |
| Ex. 4.11 | 40 | AlPO4 | CPA | 7.7 | 565 | 0.236 | 0.006 | 0.07 | 55.0 | 4.7 |
| Ex. 4.12 | 40 | SiO2 | CPA | 7.7 | 565 | 0.127 | 0.19 | 1.22 | 55.0 | 4.7 |
| Comp Ex. | 40 | AlPO4 | TAPN | 7.7 | 607 | 0.005 | | | | |
| 1.1 | | | | | | | | | | |
| Ex. 1.2 | 40 | AlPO4 | TAPN | 7.7 | 607 | 0.02 | | | 55.0 | 7.8 |
| Ex. 1.3 | 40 | AlPO4 | TAPN | 7.7 | 607 | 0.1 | 0.08 | 0.43 | 55.0 | 6.6 |
| Ex. 1.4 | 40 | AlPO4 | TAPN | 7.7 | 607 | 0.4 | 0.07 | 1.44 | 55.0 | 5.8 |
| Comp Ex. | 40 | | TAPN | 14.4 | 607 | 0.005 | | | | |
| 2.1 | | | | | | | | | | |
| Ex. 2.2 | 40 | AlPO4 | TAPN | 14.4 | 607 | 0.020 | | | 1 | 6.2 |
| Ex. 2.3 | 40 | AlPO4 | TAPN | 14.4 | 607 | 0.100 | 0.04 | 0.22 | 55.0 | 5.4 |
| Ex. 2.4 | 40 | AlPO4 | TAPN | 14.4 | 607 | 0.400 | 0.04 | 0.84 | 55.0 | 4.8 |

### Example 8.1

An ⅛: trilobed extrudate prepared as per that described in Example 3 was employed for catalyst finishing in Example 8. 50 grams of the calcined extrudate were impregnated with a solution of 190 mg of tetraammineplatinum (II) nitrate (TAPN) dissolved in 77 grams of water. In the metal incorporation the calcined extrudate was first added into a rotary impregnator and the Pt solution was added. The impregnator first cold rolled at room temperature for 1 hour, then hot rolled via steam jacket until the extrudate free rolled. The impregnated base was dried at 110° C. overnight. This sample was steam oxidized in a 3-zone furnace at 565° C. in flowing air containing 8% steam for 2 hours. The catalyst is designed as Example 8.1.

### Examples 8.2 and 8.3

Catalyst Examples 8.2 and 8.3 were prepared following the procedure described for Example 8.1 with the exception that 2.0 and 4.0 grams of ammonium nitrate, respectively, were added into TAPN impregnation solution.

### Examples 8.4 and 8.5

Catalyst Examples 8.4 and 8.5 were prepared following the procedure described for Example 8.1 with the exception that 2.0 and 4.0 grams of nitric acid, respectively, were added into TAPN impregnation solution.

### Examples 8.6 through 8.10

Examples 8.6 through 8.10 were prepared as per Examples 8.1 through 8.5 with the exception that the impregnated bases were dry oxidized in a 3-zone furnace at 565° C. in flowing air containing no steam.

### Example 9.1

An ⅛″ trilobed extrudate was prepared with 35% Versal Al₂O₃ and 65% MFI of Si/A12=40 synthesized as per Example 3. The calcined support was finished with chloroplatinic acid (CPA) using a solution to support ratio on a volumetric basis to attain a target 0.2 wt % platinum and steam oxidized at 565° C. in flowing air containing 8% steam for two hours.

### Example 9.2

25 grams of calcined support described in Example 9.1 (65% MFI-Q/35% Al₂O₃) was impregnated with a solution of 1.52 grs of CPA solution and 3.0 grs of HCl acid dissolved in 36 grs of water. The calcined support was first added to a quartz impregnator equipped with steam jacket, followed by the addition of impregnation solution. The impregnator first cold rolled at RT for one hour, then hot rolled until extrudates became free roll. The sample was dry at 110° C. in a box oven overnight. This sample was steam oxidized in a 3-zone furnace at 565° C. in flowing air containing 8% steam for 2 hours. The catalyst is designed as Example 9.2.

### Example 9.3

Example 9.3 was prepared following that described for Example 9.1 with the exception that TAPN was used in replacement of CPA.

### Example 9.4

30 grams of calcined support described in Example 9.1 (65% MFI-Q/35% Al₂O₃) was impregnated with a solution of 113 mg of TAPN and 1.2 grs of ammonium nitrate dissolved in 50 grs of water. The calcined support was first added to a quartz impregnator equipped with steam jacket, followed by the addition of impregnation solution. The impregnator first cold rolled at RT for one hour, then hot rolled until extrudates became free roll. The sample was dry at 110° C. in a box oven overnight. This sample was steam oxidized in a 3-zone furnace at 565° C. in flowing air containing 8% steam for 2 hours. The catalyst is designed as Example 9.4.

### Example 9.5

30 grams of calcined support prescribed in Example 9.1 (65% MFI-Q/35% Al₂O₃) was impregnated with a solution of 113 mg of TAPN nitrate and 2.49 grs of ammonium nitrate dissolved in 50 grs of water. The sample was dry at 110° C. in a box oven overnight. This sample was steam oxidized in a 3-zone furnace at 565° C. in flowing air containing 8% steam for 2 hours. The catalyst is designed as Example 9.5.

### Example 10

The platinum concentration profiles over the cross sections of the extrudates in Examples 8 and 9 are determined by Scanning Electron Microscopy described as follows. The catalyst sample is mounted in acrylic media and polished down into cross-section. The mount analyzed via SEM-EDS where EDS maps of each catalyst cross-section are collected. The collected EDS data for each cross section is processed into Euclidean zones of equal area from the outside edge to the center. The data from each of those zones is output as a concentration profile for each catalyst.

That data is then made into graphs, with each zone represents one point on the graph and the collection of points representing the catalyst cross-section. Catalysts described in Examples 8 and 9 exhibited varying degrees of platinum uniformity or surface enrichment. The utilization of acidity is calculated by the amount of acidity covered by 87.5% platinum from the exterior of extrudates. They are reported in Table 9 along with the available platinum surface measured essentially as per ASTM D3908-20. The amount of platinum located within the zeolite micropore is measured by the amount of CO (carbon monoxide) interacting with platinum oxides by infrared spectroscopy. In determining the platinum located within the zeolite micropores the dry and steam oxidized catalysts prepared as per Examples 8 and 9 were pre-treated at 520° C. in flowing air for 2 hours. Once cooling down in helium, CO adsorption is conducted by flowing 0.1% CO in helium over the sample for 5 minutes, equilibrating for 15 minutes and then purging with helium for 2 minutes, before taking the spectra. Peaks from about 2090 to about 2050 cm−1 are assigned as CO interacting with platinum interacting with framework oxygen within the zeolite micropores. The amount of platinum located within the zeolite micropores are reported in Table 9.

### Example 10

TABLE 9

| | | | | | | | acid | CO-Pt in |
|---|---|---|---|---|---|---|---|---|
| | | Pt- | co- | steam or | wt % | H2, | utilization | micropore, |
| Examples | binder | precursor | impreg | dry oxid | Pt | umol/gm | ( Pt SEM) | area CO/mg |
| Example 4.9 | SiO2 | CPA | none | steam | 0.152 | 1.21 | 0.767 | 0.012 |
| Example 8.1 | SiO2 | TAPN | none | steam | 0.2 | 2.46 | 0.278 | 0.021 |
| Example 8.2 | SiO2 | TAPN | NH4NO3 | steam | 0.2 | 2.28 | 0.838 | 0.021 |
| Example 8.3 | SiO2 | TAPN | NH4NO3 | steam | 0.2 | 2.58 | 0.852 | 0.030 |
| Example 8.4 | SiO2 | TAPN | HNO3 | steam | 0.2 | 2.54 | 0.676 | 0.020 |
| Example 8.5 | SiO2 | TAPN | HNO3 | steam | 0.2 | 1.75 | 0.787 | 0.016 |
| Example 8.6 | SiO2 | TAPN | none | dry | 0.2 | 4.93 | 0.257 | 0.058 |
| Example 8.7 | SiO2 | TAPN | NH4NO3 | dry | 0.2 | 6.36 | 0.791 | 0.059 |
| Example 8.8 | SiO2 | TAPN | NH4NO3 | dry | 0.2 | 6.44 | 0.852 | 0.062 |
| Example 8.9 | SiO2 | TAPN | HNO3 | dry | 0.2 | 6.26 | 0.681 | 0.077 |
| Example 8.10 | SiO2 | TAPN | HNO3 | dry | 0.2 | 6.53 | 0.799 | 0.075 |
| Example 9.1 | Al2O3 | CPA | none | steam | 0.197 | 8.64 | 0.637 | 0.012 |
| Example 9.3 | Al2O3 | TAPN | NH4NO3 | steam | 0.138 | 2.59 | 0.056 | 0.003 |
| | | | | steam | | | | |
| Example 9.4 | Al2O3 | TAPN | NH4NO3 | steam | 0.2 | 7.96 | 0.382 | 0.030 |
| Example 9.2 | Al2O3 | CPA | HCL | steam | 0.2 | 8.85 | 0.845 | 0.011 |
| Example 9.5 | Al2O3 | TAPN | NH4NO3 | steam | 0.2 | | 0.799 | |

### Example 11

Catalytic testing is conducted in a plug flow reactor of ⅞″ diameter loaded with 10 grams ⅛″ diameter trilobed extrudate using a feed blend made up of 75% isopentane and 25% cyclohexane on a weight basis at 2 hr−1 weight hourly space velocity, 2.0 hydrogen to hydrocarbon molar ratio and 300 psig pressure. The performance is measured at 420, 460, 490, 510 and 490° C. set temperatures using online GC equipped with PONA (boiling point) and Aromatics (polar) columns. Results at 490° C. set temperatures are reported in Table 10.

TABLE 10
Catalytic measurement at 488° C.

| Examples | C1 | C2 | C3 | C4 | A6-A9 | A10+ |
|---|---|---|---|---|---|---|
| Example 4.9 | 16.61 | 57.99 | 14.59 | 1.03 | 8.66 | 0.76 |
| Example 8.1 | 14.28 | 58.93 | 16.55 | 1.24 | 7.80 | 0.88 |
| Example 8.2 | 16.54 | 61.64 | 9.71 | 0.72 | 9.77 | 1.12 |
| Example 8.3 | 17.40 | 61.23 | 12.71 | 0.87 | 6.73 | 0.68 |
| Example 8.4 | 15.02 | 61.28 | 14.57 | 1.09 | 7.21 | 0.54 |
| Example 8.5 | 16.21 | 61.17 | 13.35 | 1.01 | 7.43 | 0.51 |
| Example 8.6 | 16.55 | 46.16 | 21.16 | 1.06 | 14.01 | 0.58 |
| Example 8.7 | | | | | | |
| Example 8.8 | 19.54 | 49.81 | 15.90 | 0.73 | 13.24 | 0.42 |
| Example 8.9 | 17.72 | 47.85 | 18.44 | 0.90 | 14.16 | 0.46 |
| Example 8.10 | 15.59 | 45.95 | 23.67 | 1.28 | 12.88 | 0.28 |
| Example 9.1 | 16.76 | 59.26 | 11.50 | 0.85 | 9.78 | 1.25 |
| Example 9.3 | 18.67 | 48.05 | 20.75 | 1.46 | 9.73 | 0.88 |
| Example 9.4 | 20.28 | 60.00 | 6.17 | 0.43 | 11.05 | 1.43 |
| Example 9.2 | 16.95 | 61.68 | 10.94 | 0.76 | 7.95 | 1.33 |
| Example 9.5 | 20.12 | 59.75 | 6.92 | 0.45 | 10.56 | 1.54 |

It is noted that light paraffin production to attain high ethane yields is effected by catalysts having chemisorption H₂ less than about 6 or greater about 7 umoles/gram. It is noted that ethane, and ethane and propane combined, are effected with catalysts having H₂/Pt ratios of less than 0.6 and greater 0.7. To maximize converting light paraffin and naphthene molecules into light paraffins of ethane, propane and combination of thereof, it is desired to have platinum located within zeolite micropores being less than 0.055 CO area/mg as measured by CO interacting with platinum oxide ranging from about 2080 to about 2050 cm-1 in Infrared Spectroscopy. The same observations are also noted for catalytic performance measured at 460° C. as shown in Table 11.

TABLE 11
Catalytic measurement at 460° C.

| Examples | C1 | C2 | C3 | C4 | A6-A9 | A10+ |
|---|---|---|---|---|---|---|
| Example 4.9 | 12.09 | 47.68 | 31.42 | 2.80 | 5.33 | 0.32 |
| Example 8.1 | 9.50 | 44.32 | 36.24 | 3.15 | 6.34 | 0.10 |
| Example 8.2 | 12.32 | 53.98 | 24.49 | 2.30 | 6.09 | 0.43 |
| Example 8.3 | 11.66 | 47.87 | 31.71 | 2.45 | 5.84 | 0.19 |
| Example 8.4 | 10.14 | 47.74 | 32.44 | 2.82 | 6.24 | 0.27 |
| Example 8.5 | 11.16 | 46.92 | 33.68 | 2.80 | 4.99 | 0.14 |
| Example 8.6 | 9.16 | 31.12 | 41.11 | 4.31 | 13.59 | 0.24 |
| Example 8.7 | | | 45.50 | 5.62 | 13.40 | 0.15 |
| Example 8.8 | 10.95 | 35.13 | 37.50 | 3.21 | 12.71 | 0.19 |
| Example 8.9 | 10.39 | 34.15 | 38.24 | 3.35 | 13.23 | 0.22 |
| Example 8.10 | 8.37 | 30.99 | 42.83 | 4.56 | 12.71 | 0.16 |
| Example 9.1 | 11.58 | 49.37 | 28.00 | 2.42 | 7.74 | 0.41 |
| Example 9.3 | 11.90 | 36.32 | 40.19 | 3.66 | 6.91 | 0.56 |
| Example 9.4 | 15.09 | 52.74 | 20.45 | 1.57 | 9.17 | 0.48 |
| Example 9.2 | 11.76 | 54.01 | 24.77 | 2.11 | 6.17 | 0.74 |
| Example 9.5 | 15.17 | 54.70 | 17.88 | 1.39 | 9.21 | 0.98 |

### Specific Embodiments

While the following is described in conjunction with specific embodiments, it will be understood that this description is intended to illustrate and not limit the scope of the preceding description and the appended claims.

A first embodiment of the invention is a catalyst for converting naphtha to ethane and propane wherein the catalyst contains more than 0.1 wt % platinum and has H₂ chemisorption less than about 7 umol/gm. An embodiment of the invention catalyst is a zeolitic catalyst and wherein a zeolite in said zeolitic catalyst comprises a zeolitic structure selected from the group consisting of MFI, FER, MEL, UZM-39, UZM-44, UZM-54, MWW, MFS, AEL, MSE, UZM-35 and MTW. An embodiment of the invention is one, any or all of prior embodiments in this paragraph up through the first embodiment in this paragraph wherein a catalyst has H₂ chemisorption less than about 6 umol/gm. An embodiment of the invention is one, any or all of prior embodiments in this paragraph up through the first embodiment in this paragraph wherein a catalyst has H₂ chemisorption less than about 5 umol/gm. An embodiment of the invention is one, any or all of prior embodiments in this paragraph up through the first embodiment in this paragraph wherein a catalyst has a chemisorption hydrogen to platinum ratio of less than about 0.5. An embodiment of the invention is one, any or all of prior embodiments in this paragraph up through the first embodiment in this paragraph wherein a catalyst has a chemisorption hydrogen to platinum ratio of less than about 0.45. An embodiment of the invention is one, any or all of prior embodiments in this paragraph up through the first embodiment in this paragraph wherein the catalyst has a chemisorption hydrogen to platinum ratio of less than about 0.35. An embodiment of the invention is one, any or all of prior embodiments in this paragraph up through the first embodiment in this paragraph wherein the catalyst has more than about 0.15 mmol/gram acidity. An embodiment of the invention is one, any or all of prior embodiments in this paragraph up through the first embodiment in this paragraph wherein the catalyst contains about 0.2 wt % platinum.

A second embodiment of the invention is a catalyst for converting naphtha to ethane and propane wherein the catalyst comprises at least 0.1 wt % platinum and has a chemisorption hydrogen to platinum ratio of less than about 0.5. An embodiment of the invention is one, any or all of prior embodiments in this paragraph up through the second embodiment in this paragraph wherein the catalyst is a zeolitic catalyst and wherein a zeolite in said zeolitic catalyst comprises a zeolitic structure selected from the group consisting of MFI, FER, MEL, UZM-39, UZM-44, UZM-54, MWW, MFS, AEL, MSE, UZM-35 and MTW. An embodiment of the invention is one, any or all of prior embodiments in this paragraph up through the second embodiment in this paragraph wherein the catalyst comprises about 0.2 wt % platinum. An embodiment of the invention is one, any or all of prior embodiments in this paragraph up through the second embodiment in this paragraph wherein the catalyst has a chemisorption hydrogen to platinum ratio of less than about 0.35. An embodiment of the invention is one, any or all of prior embodiments in this paragraph up through the second embodiment in this paragraph wherein wherein the catalyst has more than about 0.12 mmol/gram acidity. An embodiment of the invention is one, any or all of prior embodiments in this paragraph up through the second embodiment in this paragraph wherein the catalyst has more than about 0.15 mmol/gram acidity.

A third embodiment of the invention is a process for converting naphtha comprising: contacting a naphtha stream with a zeolitic catalyst wherein the catalyst contains more than 0.1 wt % platinum and has H₂ chemisorption less than about 6 umol/gm at conditions which dehydrogenate paraffins to olefins, interconvert the olefins to lighter olefins and hydrogenate the lighter olefin to produce a light paraffin stream comprising more than 80% ethane and propane at an ethane to propane ratio of greater than about 0.5. An embodiment of the invention is one, any or all of prior embodiments in this paragraph up through the third embodiment in this paragraph wherein the catalyst is a zeolitic catalyst and wherein a zeolite in said zeolitic catalyst comprises a zeolitic structure selected from the group consisting of MFI, FER, MEL, UZM-39, UZM-44, UZM-54, MWW, MFS, AEL, MSE, UZM-35 and MTW. An embodiment of the invention is one, any or all of prior embodiments in this paragraph up through the third embodiment in this paragraph wherein the catalyst has a chemisorption hydrogen to platinum ratio of less than about 0.5. An embodiment of the invention is one, any or all of prior embodiments in this paragraph up through the third embodiment in this paragraph wherein the catalyst has more than about 0.12 mmol/gram acidity. An embodiment of the invention is one, any or all of prior embodiments in this paragraph up through the third embodiment in this paragraph wherein the catalyst has more than about 0.15 mmol/gram acidity. An embodiment of the invention is one, any or all of prior embodiments in this paragraph up through the third embodiment in this paragraph wherein the light paraffin stream comprises about 55% ethane. An embodiment of the invention is one, any or all of prior embodiments in this paragraph up through the third embodiment in this paragraph wherein the pressure ranges from about 0.1 to about 3 MPa, weight space velocity ranges from about 0.3 to about 20 hr⁻¹ and the temperatures ranges from about 350 to about 600° C.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A catalyst for converting naphtha to ethane and propane wherein the catalyst contains more than 0.1 wt % platinum and has H₂ chemisorption less than about 6 or greater than about 7 umol/gm.

2. The catalyst of claim 1 wherein said catalyst is a zeolitic catalyst and wherein a zeolite in said zeolitic catalyst comprises a zeolitic structure selected from the group consisting of MFI, FER, MEL, UZM-39, UZM-44, UZM-54, MWW, MFS, AEL, MSE, UZM-35 and MTW.

3. The catalyst of claim 1 wherein said catalyst has chemisorption H₂ to platinum ratios being less than about 0.6 or greater than about 0.7.

4. The catalyst of claim 1 wherein said catalyst has H₂ chemisorption less than about 5 or greater than 7 umol/gm.

5. The catalyst of claim 1 wherein said catalyst has a chemisorption hydrogen to platinum ratio of less than about 0.5 or greater than 0.75.

6. The catalyst of claim 1 wherein said catalyst has a chemisorption hydrogen to platinum ratio of less than about 0.45 or greater than 0.8.

7. The catalyst of claim 1 wherein said catalyst has a chemisorption hydrogen to platinum ratio of less than about 0.35 or greater than 0.8.

8. The catalyst of claim 1 wherein said catalyst has more than about 0.12 mmol/gram acidity.

9. The catalyst of claim 1 wherein said catalyst contains greater than about 0.1 wt % platinum.

10. A catalyst for converting naphtha to ethane and propane wherein the catalyst comprises at least 0.1 wt % platinum and has a chemisorption hydrogen to platinum ratio of less than about 0.5.

11. The catalyst of claim 10 wherein said catalyst is a zeolitic catalyst and wherein a zeolite in said zeolitic catalyst comprises a zeolitic structure selected from the group consisting of MFI, FER, MEL, UZM-39, UZM-44, UZM-54, MWW, MFS, AEL, MSE, UZM-35 and MTW.

12. The catalyst of claim 10 comprising greater than about 0.1 wt % platinum.

13. The catalyst of claim 10 wherein the catalyst has a chemisorption hydrogen to platinum ratio of less than about 0.35 and greater than about 0.75.

14. The catalyst of claim 10 wherein said catalyst has more than about 0.12 mmol/gram acidity.

15. The catalyst of claim 10 wherein the acid utilization is greater than about 25% and preferably greater than about 40% acid utilization.

16. A process for converting naphtha comprising:
contacting a naphtha stream with a zeolitic catalyst wherein the catalyst contains more than 0.1 wt % platinum and has H₂ chemisorption less than about 6 umol/gm at conditions which dehydrogenate paraffins to olefins, interconvert the olefins to lighter olefins and hydrogenate the lighter olefin to produce a light paraffin stream comprising more than about 75% ethane and propane at an ethane to propane ratio of greater than about 0.5.

17. The process of claim 16 wherein said H₂ chemisorption is less than about 5 umol/gram.

18. The process of claim 16 wherein said catalyst is a zeolitic catalyst and wherein a zeolite in said zeolitic catalyst comprises a zeolitic structure selected from the group consisting of MFI, FER, MEL, UZM-39, UZM-44, UZM-54, MWW, MFS, AEL, MSE, UZM-35 and MTW.

19. The process of claim 16 wherein said catalyst of which the platinum located within the zeolite micropore is less than about 0.055 CO area/mg measured by Infrared Spectroscopy.

20. The process of claim 16 wherein said catalyst has more than about 0.12 mmol/gram acidity.
